(19) United States
(12) Patent Application Publication
(10) Pub. No.: US 2004/0057947 A1
(43) Pub. Date: Mar. 25, 2004

(54) Drug combinations based on magnesium salts and fibrinolytics

(75) Inventors: Hermann Duettmann, Gau-Algesheim (DE); Judith Duettmann, Gau-Algesheim (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/601,205

(22) Filed: Jun. 20, 2003

Related U.S. Application Data
(60) Provisional application No. 60/408,145, filed on Sep. 4, 2002.

(30) Foreign Application Priority Data
Jul. 9, 2002 (DE) 102 30 752

(51) Int. Cl. A61K 38/48; A61K 31/19; A61K 33/06; A61K 33/14

(52) U.S. Cl. 424/94.64; 514/561; 514/557; 424/681; 424/682

(57) ABSTRACT

The invention relates to new drug combinations based on magnesium salts 1 and fibrinolytics 2, processes for the preparation thereof as well as the use thereof for preparing pharmaceutical compositions for the treatment of ischaemic conditions.

The invention relates to new drug combinations based on magnesium salts 1 and fibrinolytics 2, processes for the preparation thereof as well as the use thereof for preparing pharmaceutical compositions for the treatment of ischaemic conditions.

### DESCRIPTION OF THE INVENTION

The invention relates to drug combinations containing one or more, preferably one magnesium salt 1 and one or more, preferably one fibrinolytic 2, optionally in the presence of conventional excipients or carriers.

A) Magnesium Salts 1 Which may be Used According to the Invention:

Within the scope of the present invention preferred magnesium salts 1 are those selected from the group consisting of magnesium adipate, magnesium-L-aspartate, magnesium carbonate, magnesium-L-hydrogen aspartate, magnesium hydrogen citrate, magnesium hydrogen glutamate, magnesium sulphate, magnesium chloride, trimagnesium dicitrate and magnesium acetate.

Particularly preferred within the scope of the present invention are the magnesium salts 1 selected from the group consisting of magnesium sulphate, magnesium chloride and magnesium acetate, while magnesium sulphate is of particular importance according to the present invention.

B) Fibrinolytics 2 Which may be Used According to the Invention:

Within the scope of the present invention the fibrinolytics 2 selected from among the plasminogen activators are preferred. Of particular interest are alteplase (human tissue plasminogen activator, t-PA), tenecteplase, reteplase, streptokinase, urokinase, anistreplase, monteplase, nateplase, duteplase, lanoteplase, silteplase, amediplase and desmoteplase. All these fibrinolytics are known in the art.

Alteplase (amino acid sequence: GenBank Accession No. AAB59510) is a fibrinolytic licensed for use as a drug, the preparation of which by recombinant expression, preferably in cell lines of the Chinese hamster Cricetulus griseus (CHO cells), as well as its pharmaceutical formulation and medicinal use have been described in detail in the prior art (Pennica et al., Nature 301, 214-221 (1983); EP 0 093 619; Andersen et al., Biotechnol Bioeng 70 (1), 25-31 (2000); Dowd et al., Biotechnol Prog 16 (5), 786-794 (2000); Fann et al., Biotechnol Bioeng 69 (2), 204-212 (2000); Werner et al., Arzneimittelforschung 48 (8), 870-880 (1998); Wernicke et Will, Anal Biochem 203 (1), 146-150 (1992); Bos et al., Biochim Biophys Acta 1117 (2), 188-192 (1992); Dodd et al., FEBS Lett 209 (1), 13-17 (1986); Matsuo et al., J Chromatogr 369 (2), 391-397 (1986); Einarsson et al., Biochim Biophys Acta 830 (1), 1-10 (1985); Kruithof et al., Biochem J 226 (3), 631-636 (1985); Nguyen et al., Pharm Biotechnol. 5, 91-134 (1993); The Gusto III Investigators, N Engl J Med. Oct. 16, 1997;337(16):1118-23; EP 0 239 292; WO 86/05514).

Tenecteplase (TNK-tPA; T103N,N117Q,KHRR(296-299)AAAA-tPA) is also a fibrinolytic licensed for use as a drug. Its preparation and use is described in detail in the references WO 93/24635; Keyt et al., Proc Natl Acad Sci USA. Apr. 26, 1994;91(9):3670-4; Turcasso et Nappi, Ann Pharmacother. 2001 October;35(10):1233-40; MacGahan, Issues Emerg Health Technol. 2001 January;(13):1-6; Davydov et Cheng, Clin Ther. 2001 July;23(7):982-97; The Assent II investigators, Lancet. Aug. 28, 1999;354(9180):716-22.

The preparation of reteplase, which is also licensed for drug use, and its pharmaceutical formulations and uses are described in the literature in WO 90/03497; WO 91/08765, WO 91/08766, and Noble et McTavish, Drugs. 1996 October; 52(4):589-605. The preparation, pharmaceutical formulation and medical use of lanoteplase are described in the literature in WO 87/04722 and WO 90/08557. The preparation, pharmaceutical formulation and medical use of desmoteplase are described in the literature in WO 90/09438 and WO 97/29188. The preparation, pharmaceutical formulation and medical use of anistreplase, which is licensed for drug use, is described in the literature in EP 0 028 489, Fears, Semin Thromb Hemost 15 (2), 129-139 (1989); Anderson et al., Circulation. 1991 January;83(1):126-40; Been et al., Int J Cardiol. 1986 April;11(1):53-61; Marder et al., Ann Intern Med. 1986 March;104(3):304-10; Walker et al, Thromb Haemost. Apr. 30, 1984;51(2):204-6, and Matsuo et al, Thromb Res Suppl. Nov. 15, 1981;24(4):347-58. Urokinase is described in EP 0 143 949, EP 0 154 272, EP 0 303 028, and EP 0 620 279.

A large number of other plasminogen activators which may be used as component 2 for the combination according to the invention are described in the literature.

Particularly preferred within the scope of the present invention are the fibrinolytics 2 selected from the group consisting of alteplase (t-PA), tenecteplase, reteplase, streptokinase, urokinase, anistreplase, monteplase and nateplase. Particularly preferred fibrinolytics 2 according to the invention are selected from among alteplase (t-PA), tenecteplase, reteplase, urokinase and anistreplase, while alteplase, tenecteplase and reteplase and most preferably alteplase are of exceptional importance according to the invention.

C) Use of the Drug Combinations of 1 and 2 According to the Invention:

The present invention further relates to the use of the combinations according to the invention of one or more, preferably one magnesium salt 1 and one or more, preferably one fibrinolytic 2 for preparing a pharmaceutical composition for the treatment of ischaemic conditions of various origins. Preferably, the present invention relates to the use of the combinations according to the invention of one or more, preferably one magnesium salt 1 and one or more, preferably one fibrinolytic 2 for preparing a pharmaceutical composition for the treatment of cardiac or cerebral ischaemias, most preferably for the treatment of stroke. Of particular importance within the scope of the present invention is the use of the combinations according to the invention of one or more, preferably one magnesium salt 1 and one or more, preferably one fibrinolytic 2 for the treatment of ischaemic stroke, most preferably acute ischaemic stroke.

The present invention further relates to a process for treating ischaemic conditions of various origins which is characterised in that a combination according to the invention of one or more, preferably one magnesium salt 1 and one or more, preferably one fibrinolytic 2 is administered. The present invention preferably relates to a method of treating cardiac or cerebral ischaemias, most preferably stroke, and more preferably according to the invention ischaemic stroke, most preferably acute ischaemic stroke, which is characterised in that a combination according to the invention of one or more, preferably one magnesium salt 1 and one or more, preferably one fibrinolytic 2 is administered.

The present invention further relates to the use of one or more, preferably one magnesium salt 1 for preparing a pharmaceutical composition for the combined treatment of ischaemic conditions of various origins with one or more, preferably one fibrinolytic 2. The present invention preferably relates to the above-mentioned use for preparing a pharmaceutical composition for the combined treatment of cardiac or cerebral ischaemias, most preferably for the treatment of stroke with one or more, preferably one fibrinolytic 2. Of particular importance within the scope of the present invention is the present use for the combined treatment of ischaemic stroke, most preferably acute ischaemic stroke with one or more, preferably one fibrinolytic 2.

The present invention further relates to a method of treating ischaemic conditions of various origins which is characterised in that one or more, preferably one magnesium salt 1 and one or more, preferably one fibrinolytic 2 are administered simultaneously or sequentially in one single or two separate, preferably in two separate preparations. The present invention preferably relates to a method of treating cardiac or cerebral ischaemias, most preferably stroke, and more preferably according to the invention ischaemic stroke, most preferably acute ischaemic stroke, which is characterised in that one or more, preferably one magnesium salt 1 and one or more, preferably one fibrinolytic 2 are administered simultaneously or sequentially in one single or two separate, preferably in two separate preparations.

D.1) Administration of the Drug Combinations of 1 and 2 According to the Invention:

The drug combinations according to the invention may contain the active ingredients 1 and 2 in one single or two separate preparations. In the combinations of magnesium sulphate, magnesium chloride or magnesium acetate as component 1 with alteplase (t-PA), tenecteplase, reteplase, urokinase or anistreplase as component 2 which are of particular importance according to the invention, the two components are preferably contained in two separation preparations, for example in the form of a so-called kit. Separate formulations of the two components 1 and 2 are described in detail in the following paragraphs.

The combination of 1 and 2 according to the invention may be administered, within the scope of the abovementioned use and within the scope of the above-mentioned process, by simultaneously administering the combination of 1 and 2 or, when 1 and 2 are present in different preparations, by administering components 1 and 2 simultaneously or sequentially. The term sequentially within the scope of the present invention refers to any method of administering components 1 or 2 which does not take place simultaneously. By simultaneous administration is meant the method of administration in which at least one of components 1 and 2 is administered for example by infusion over a longer period and the other component is also used during this period. If the two components 1 and 2 are both administered by infusion over a longer period of time, the word simultaneously for the purposes of the present invention means that the infusion periods overlap for at least a short time.

Particularly when treating ischaemic stroke, which is the preferred indication within the scope of the present invention, most preferably when treating acute ischaemic stroke, components 1 and 2 are preferably given simultaneously or at least within a short time of each other, i.e. for example within one hour. Treatment with the drug combinations according to the invention is particularly effective when it is given as quickly as possible after the stroke takes place. Preferably, the treatment starts at the latest within about 5 hours, most preferably within 4 hours, more preferably still within 3 hours after the stroke occurs.

D.2) Pharmaceutical Formulation and Administration of Component 1:

The magnesium salt 1 used within the scope of the combination according to the invention may be given orally or parenterally within the scope of the present invention, parenteral administration being particularly preferred. It may be administered parenterally particularly by intravenous, intraarterial, intramuscular, intra- or subcutaneous injection. Typical formulations are aqueous solutions for infusion or injection, which may optionally contain conventional stabilisers, solubilisers and preservatives as further ingredients.

Typically, within the scope of the present invention, a total of between 30 and 120 mmol, preferably about 50 to 100 mmol, more preferably about 70 to 90 mmol of magnesium are administered per dose. The substance is administered, for example in the form of an infusion which is administered over a period of about 6 to 48 hours, preferably about 12 to 36 hours, more preferably about 18 to 30 hours. Within this period the dose in question can be varied for each time interval. For example in a first interval between about 5 and 25 mmol, preferably between about 10 and 20 mmol of magnesium may be administered over a period of about 5 minutes to 1 hour, preferably over a period of about 10 to 30 minutes and then in a second interval between 25 and 100 mmol, preferably between about 40 and 80 mmol, more preferably between about 50 and 70 mmol of magnesium are administered, for example, over a period of about 5 to 48 hours, preferably about 12 to 36 hours, more preferably about 20 to 28 hours. However, the dosage and administration period may differ from those given above as a guide, depending on the patient and the clinical picture. As a rule it may be desirable to adjust the dosage and period of administration of magnesium so that the plasma levels of magnesium thus produced are above the natural plasma levels of magnesium by about a factor of 1.5 to 2.5, preferably by about a factor of 2. For example, component 1 may also be administered by one or more injections.

D.2.1) Examples of Pharmaceutical Formulations of Component 1:

| | |
|---|---|
| a) Injectable solution: | |
| magnesium sulphate | 1000 mg |
| water for injections 10 ml | |
| b) Injectable solution: | |
| magnesium sulphate | 2000 mg |
| water for injections 10 ml | |
| c) concentrated solution for infusion (for | |
| preparing a solution for infusion): | |
| magnesium sulphate | 5000 mg |
| water for injections 10 ml | |

In the formulation examples shown above the information relates to magnesium sulphate in the anhydrous form.

D.3) Pharmaceutical Formulation and Administration of Component 2:

The fibrinolytic 2 used within the scope of the drug combination according to the invention is generally a polypeptide which has to be given parenterally. It may be given particularly by intravenous, intraarterial, intramuscular, intra- or subcutaneous injection, but may also be administered by inhalation of a powder or aerosol. Typical formulations are freeze-dried preparations (lyophilisates) of the polypeptide, which are reconstituted immediately before administration with a solution for injection or infusion. The reconstituting solution may be water or a buffered aqueous solution. The formulation may, however, also consist of an aqueous solution which is preferably buffered with a physiologically acceptable buffer and may additionally contain conventional stabilisers, solubilisers and preservatives. Examples of conventional adjuvants for liquid or solid formulations of this kind are alkali metal hydrogen phosphate/alkali metal dihydrogen—phosphate, sodium chloride, serum albumin, polyoxyethylenesorbitan-monolaurate (Tween® 20), polyoxyethylenesorbitan-monooleate (Tween® 80), ethylenediamine-tetraacetate (EDTA), sucrose, mannitol, dextran, amino acid and benzyl alcohol (the latter only for liquid formulations). They are generally administered parenterally, preferably by intravenous injection or infusion. The mode of application and dosage preferably depend on the fibrinolytic selected, particularly its specific biological activity and half-life in the blood plasma. Thus, alteplase, which has a relatively short half-life, is typically administered in a total dosage of 100 mg as follows: 10-15 mg as an intravenous bolus, followed by an intravenous infusion of 50 mg over a period of 30 to 60 minutes, followed by another infusion of 60-180 minutes up to the maximum dose. Tenecteplase has a longer half-life and can therefore be administered as a single bolus, based on body weight, up to a maximum dose of 50 mg. Reteplase, which has a moderate half-life and low specific activity, is administered as an intravenous double bolus at an interval of 30 minutes in a dosage of 10 units (560 mg) per bolus. The skilled man knows how to determine the correct dosage for a new pharmaceutical composition. For the plasminogen activators mentioned by name, the skilled man can find information as to formulations and dosages in the literature mentioned above.

D.3.1) Pharmaceutical Formulation Examples of Component 2:

Formulations of component 2 are known in the prior art and may be obtained commercially. Some commercially available formulations which may be used according to the invention are mentioned below by way of example and as an illustration.

Alteplase: (powder and solvent for preparing a solution for injection/infusion)

| | |
|---|---|
| Composition: | Alteplase 10 mg/20 mg/50 mg/100 mg. |
| Other ingredients: | Arginine, phosphoric acid, Polysorbate 80. |
| | water for injections. |

Tenecteplase: (powder and solvent for preparing an injectable solution)

| | |
|---|---|
| Composition: | Tenecteplase 8000 U/10 000 U (40 mg/50 mg). |
| Other ingredients: | Arginine, phosphoric acid, Polysorbate 20. |
| | water for injections 8 ml/10 ml. |

Reteplase: (powder and solvent for preparing an injectable solution)

| | |
|---|---|
| Composition: | Reteplase 0.56 g (corresponds to 10 units). |
| Other ingredients: | Tranexamic acid, potassium monohydrogen phosphate, |
| | phosphoric acid, sucrose, polysorbate 80. |
| | water for injections 10 ml. |

Streptokinase: (Dry substance for a solution for infusion)

| | |
|---|---|
| Composition: | highly-purified streptokinase 250000 I.U./750000 I.U./ |
| | 1500000 I.U. as dry substance. |
| Other ingredients: | Human albumin, sodium-L-hydrogen |
| | glutamate 1H₂O, Polygelin. |

Streptokinase: (oral tablets)

| | |
|---|---|
| Composition: | Streptokinase 10,000 I.U., Streptodornase 2500- |
| | 10,000 I.U. |
| Other ingredients: | Magnesium stearate, calcium hydrogen phosphate, |
| | maize starch, gum arabic. |

Urokinase: (dry substance)

| | |
|---|---|
| Composition: | Urokinase 500,000 I.U. |
| Other ingredients: | sodium monohydrogen phosphate, sodium dihydrogen |
| | phosphate, human albumin. |

Urokinase: (dry substance)

| | |
|---|---|
| Composition: | Urokinase (human) 500,000 I.U. |
| Other ingredients: | sodium dihydrogen phosphate, sodium monohydrogen |
| | phosphate, sodium chloride, Dextran 40. |

Anistreplase: (dry substance and solvent for i.v. injection)

| | |
|---|---|
| Composition: | 209-230 mg of dry substance with anistreplase 29.55- |
| | 30.03 mg. |
| Other ingredients: | 4-amidinophenyl (p-anisate)-HCl 0.15-0.17 mg, |
| | dimethylsulphoxide 1-2 mg, aminocaproic acid 1.2- |
| | 1.6 mg, D-mannitol, human albumin, lysine-1HCl, |
| | sodium hydroxide, glycerol, water for injections. |

What is claimed is:

1) A drug combination comprising one or more magnesium salts and one or more fibrinolytics.

2) The drug combination as recited in claim 1, which is contained in a single formulation.

3) The drug combination as recited in claim 1 wherein the magnesium salt is selected from the group consisting of magnesium adipate, magnesium-L-aspartate, magnesium carbonate, magnesium-L-hydrogen aspartate, magnesium hydrogen citrate, magnesium hydrogen glutamate, magnesium sulphate, magnesium chloride, trimagnesium dicitrate and magnesium acetate.

4) The drug combination as recited in claim 3 wherein the magnesium salt is selected from the group consisting of magnesium sulphate, magnesium chloride and magnesium acetate.

5) The drug combination as recited in claim 1 wherein the fibrinolytic is selected from among the plasminogen activators.

6) The drug combination as recited in claim 5 wherein the fibrinolytic is selected from the group consisting of alteplase (human tissue plasminogen activator, t-PA), tenecteplase, reteplase, streptokinase, urokinase, anistreplase, monteplase, nateplase, duteplase, lanoteplase, silteplase, amediplase and desmoteplase.

7) The drug combination as recited in claim 1 wherein the magnesium salt or salts and the fibrinolytic or fibrinolytics are in separate formulations.

8) A method for treating ischemic conditions in a warm-blooded animal which comprises administering a therapeutically effective amount of the drug combination as recited in claim 1.

9) A method for treating cardiac ischemia in a warm-blooded animal which comprises administering a therapeutically effective amount of the drug combination as recited in claim 1.

10) A method for treating cerebral ischemia in a warm-blooded animal which comprises administering a therapeutically effective amount of the drug combination as recited in claim 1.

11) A method for treating stroke in a warm-blooded animal which comprises administering a therapeutically effective amount of the drug combination as recited in claim 1.
